(19) United States
(12) Patent Application Publication
(10) Pub. No.: US 2007/0191788 A1
(43) Pub. Date: Aug. 16, 2007

(54) Pre-primed IV tubing

(76) Inventor: Timothy K. Volskay, Punta Gorda, FL (US)

Correspondence Address: EDWARD M. LIVINGSTON, PA, 963 TRAIL TERRACE DRIVE, NAPLES, FL, 34103, US

(21) Appl. No.: 11/355,582

(22) Filed: Feb. 16, 2006

(51) Int. Cl. A61M 5/00

(52) U.S. Cl. 604/257; 604/264

(57) ABSTRACT

A pre-primed IV tubing (1) having a lumen (5) and first and second ends (2) and (3). The lumen (5) of the pre-primed IV tubing (1) is completely filled with solution (4), devoid of air and is capped at both ends (2) and (3). The pre-primed IV tubing (1) may be packaged in a sterile bag (8) or stored in a container designed for storage and transport of intravenous fluids and medications. Alternatively, the pre-primed IV tubing (1) may be spiked or otherwise joined to a pre-filled IV solution bag (7) to form an IV delivery system. The pre-primed IV tubing 1 may contain the same solution (4) as held in the IV solution bag (7) or any other compatible solution (4). Furnishing the pre-primed IV tubing (1) with a universal solution eliminates the tedious process of expelling air from the tubing and speeds setup of an IV delivery system.

### CROSS REFERENCE TO RELATED APPLICATION

This application claims the benefit of U.S. Provisional Application filed Mar. 16, 2005.

### BACKGROUND OF THE INVENTION

This invention relates to medical tubing, more particularly, a pre-primed IV tubing for intravenous (IV) fluid therapy.

Maintaining sanitary and sterile conditions are a must in the medical field. The utmost care must be taken when treating a patient, especially when intravenously injecting a patient with medicine or when administering fluids to a patient by intravenous infusion. Properly injecting medicine or infusing medication, nutrients and electrolytes into the patient with an intravenous fluid delivery system requires practice and skill. The intravenous fluid (IVF) delivery system must be prepared by connecting the intravenous (IV) tubing to an IV solution bag. The IV bag is spiked with an IV tube and the solution within the IV bag is drawn out through the IV tubing in order to displace or push out any air found within the lumen of the IV tube. The technique of drawing solution completely through the IV tubing is called priming. It is imperative that the IV tubing be primed in order to preen the possibility of an air embolus from being introduced into the patient's bloodstream and ultimately obstructing blood circulation, potentially resulting in a fatal embolism.

The IV bag is not properly primed when air is present in the lumen of the IV tubing. If improperly primed, the IV tubing must be re-primed by first disconnecting the IV tube from the patient and then drawing a solution a second time through the lumen of the IV tubing until the remaining air is removed. Re-priming an IVF delivery system is both time consuming and potentially dangerous. During the re-priming process, the nurse must take the time to disconnect the attached tubing from the patient. At this point, the tip that was inserted directly into the patient's IV access site is exposed to the surrounding environment. If the tip, at any time, touches any non-sterile surface, such as a bed sheet, a stretcher side rail or the patient's skin, it is considered contaminated.

Unfortunately, contamination can occur without the nurse knowing it. For example, during an emergency when nurses are moving about in a hurry, it would be easy to unknowingly contaminate the IV tubing tip during the re-priming process. Ultimately, this means a nurse could accidentally (and easily) introduce an unsafe microorganism or bacteria into the patient's bloodstream.

Additionally, the time needed to re-prime the tubing is time that could be used to tend to a patient's needs. Precious time is lost when a nurse has to re-prime a patient's IV tubing, especially during an emergency, when seconds count.

Finally, because a predetermined volume of solution is completely drawn through the IV tubing in order to properly prime an IV tube, this predetermined volume of solution is wasted as it is not delivered into the patient.

Thus, a need exists for a device that eliminates the need to prime and/or re-prime IV tubing.

The relevant prior art includes the following references:

| U.S. Pat. No. | | Issue/ |
|---|---|---|
| (U.S. unless stated otherwise) | Inventor | Publication Date |
| 2003/0130624 | Kowalik et al. | Jul. 10, 2003 |
| 4,187,847 | Loeser | Feb. 12, 1980 |
| 4,678,460 | Rosner | Jul. 07, 1987 |
| 5,211,201 | Kamen et al. | May 18, 1993 |
| 5,382,161 | Roane | Jan. 17, 1995 |
| 5,713,865 | Manning et al. | Feb. 03, 1998 |
| 5,935,105 | Manning et al. | Aug. 10, 1999 |
| 6,156,025 | Niedospial, Jr. et al. | Dec. 05, 2000 |
| 5,843,035 | Bowman et al. | Dec. 01, 1998 |
| 6,213,978 | Voyten | Apr. 10, 2001 |
| 6,290,503 | Lemon et al. | Sep. 18, 2001 |
| EP 0220551 | Muhlbauer | May 06, 1987 |
| EP 0293591 | Abbot Laboratories | Dec. 07, 1988 |

In addition, the prior art also includes www.bbraunusa.com a website featuring a system to simplify intravenous antibiotic delivery to patients.

### SUMMARY OF THE INVENTION

The primary object of the present invention is to provide pre-primed IV tubing that is sterile.

A further object of the present invention is to provide pre-primed IV tubing that is easy to use.

An even further object of the present invention is to provide pre-primed IV tubing that is sanitary.

Another object of the present invention is to provide pre-primed IV tubing that reduces the likelihood of solution spillage/loss.

The present invention fulfills the above and other objects by providing pre-primed IV tubing having a lumen and two ends. The lumen of the IV tubing is completely filled, preferably with a universally compatible intravenous solution such as saline, thereby displacing all the air between the two ends wherein the two ends are capped. The pre-primed IV tubing may be manufactured by itself, prepared for spiking to the IV solution bag. The pre-primed IV tubing is preferably packaged in a sterile protective material wherein a user simply removes the pre-primed IV tubing from the protective packaging and spikes an IV solution bag with one end of the pre-primed IV tubing.

Alternatively, the pre-primed IV tubing may be furnished in combination with a pre-filled IV solution bag, the tubing already being pre-spiked into the IV bag. The pre-primed IV tubing may contain the same solution as that held in the IV solution bag.

The above and other objects, features and advantages of the present invention should become even more readily apparent to those skilled in the art upon a reading of the following detailed description in conjunction with the drawings wherein there is shown and described illustrative embodiments of the invention.

### BRIEF DESCRIPTION OF THE DRAWINGS

In the following detailed description, reference will be made to the attached drawings in which:

FIG. 1 is a frontal plan view of un-primed IV tubing according to the prior art;

FIG. 2; is a frontal plan view of a pre-primed IV tubing of the present invention in a sterile package and

FIG. 3 is a frontal view of an alternate embodiment of a pre-primed IV tubing of the present invention.

### DESCRIPTION OF THE PREFERRED EMBODIMENTS

For purposes of describing the preferred embodiment, the terminology used in reference to the numbered components in the drawings is as follows:

| | |
|---|---|
| 1. | pre-primed IV tubing, generally |
| 2. | first end |
| 3. | second end |
| 4. | solution |
| 5. | lumen |
| 6. | un-primed IV tubing |
| 7. | IV solution bag |
| 8. | packaging |
| 9. | access port |
| 10. | drip chamber |
| 11. | caps |

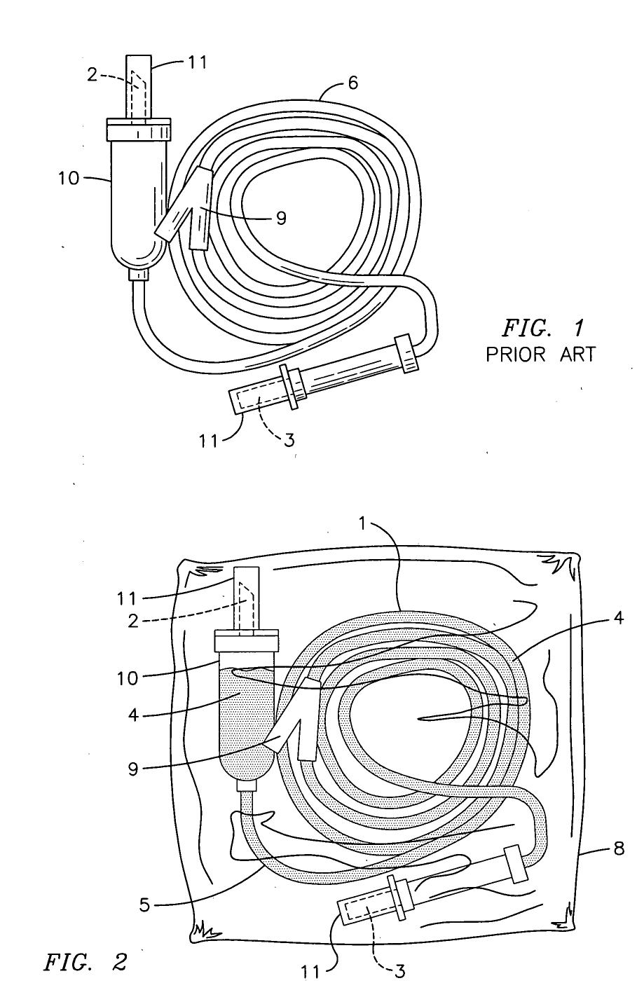

With respect to FIG. 1, a frontal plan view of un-primed IV tubing 6 according to the prior art is shown. The conventional un-primed IV tubing 6 has a first end 2 and a second end 3, both ends 2 and 3 of which have caps 11. The un-primed IV tubing 6 is currently utilized in the medical field in preparing an IV fluid delivery system. In order to prepare the IV tubing 6, the tubing 6 must be joined to an IV solution bag 7 by spiking the first end 2 of the IV tubing 6 into the IV solution bag 7. Once the IV solution bag 7 is spiked, the IV tubing 6 is primed by drawing the solution 4 contained in the IV solution bag 7 completely through the IV tubing 6. Proper priming is attained when the lumen 5 of the IV tubing 6 is entirely filled with the solution 4 of the IV solution bag 7. Proper priming prevents an air embolus from being introduced into the patient's blood circulation system, which may prove fatal to the patient.

On the other hand, the pre-primed IV tubing 1 of the present invention proves to be a safer alternative to the conventional un-primed IV tubing 6 as shown in a frontal plan view of a pre-primed IV tubing 1 of the present invention in FIG. 2. The IV tubing 1 is of a predetermined length and contains a predetermined solution 4, which is preferably universally compatible, such as a saline solution. The pre-primed IV tubing 1 has a first end 2 and a second end 3, each of both ends being capped 11 to retain the solution 4 within the lumen 5 of the tubing 1. The pre-primed IV tubing 1 is completely filled with a predetermined volume of solution 4 so as to expel air bubbles from the IV tubing 1. The lumen 5 of the pre-primed IV tubing 1 is completely devoid of air and only contains the solution 4. The pre-primed IV tubing 1 is ready to be spiked into an IV solution bag 7 containing medication, nutrient, electrolyte, blood or other solutions 4 for administration.

The pre-primed IV tubing 1 of the present invention preferably contains a universally compatible solution 4, such as normal saline. The pre-primed IV tubing 1 is preferably delivered to medical facilities, medical distributors and the like in specialized storage containers, such as refrigerated containers or may be packaged in sterile packaging 8, as shown in FIG. 2, or other packaging materials. A user removes the pre-primed IV tubing 1 from the sterile packaging 8 and spikes an IV solution bag 7 to prepare an IV fluid delivery system.

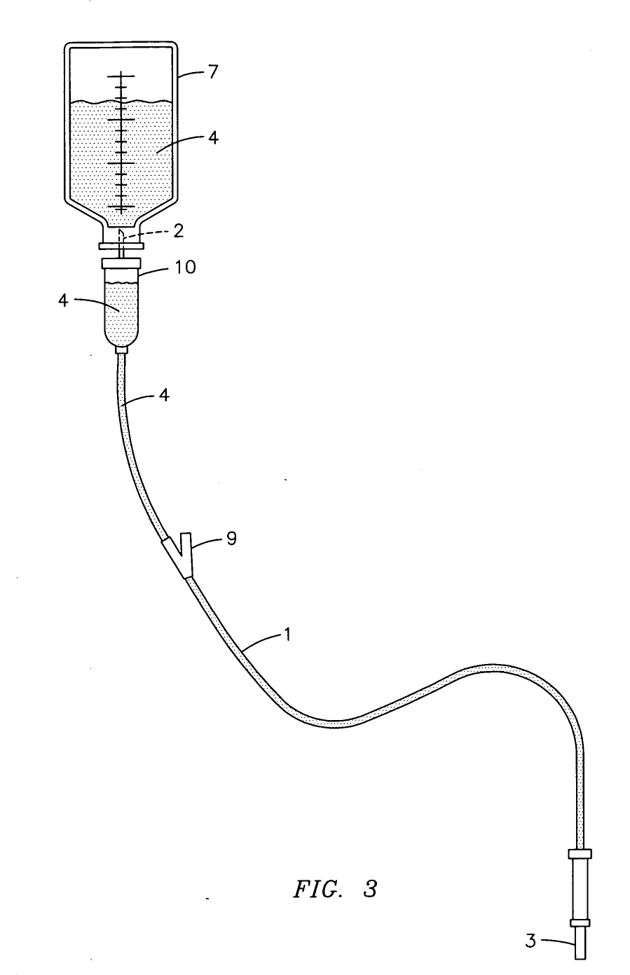

Finally, FIG. 3 shows a frontal view of an alternate embodiment of the pre-primed IV tubing 1 of the present invention wherein the pre-primed IV tubing 1 is pre-spiked into the IV solution bag 7 which preferably contains medication or intravenous solution 4. The predetermined solution 4 stored in the pre-primed IV tubing 1 may be the same as the predetermined solution 4 disposed in the IV solution bag 7 or a different, but compatible, predetermined solution 4. The use of the alternate embodiment of the pre-primed IV tubing 1 permits rapid and quick setup of an IV system, even by inexperienced individuals in emergency situations.

The pre-primed IV tubing 1 uses conventional IV tubing, which is well known in the prior art. The IV tubing may include an access port 9 for administering medication by injection through the access port 9 and infusion through the IV tubing. The IV tubing may also include a drip chamber 10 adjacent the first end 2. The access port 9 may be conventional access ports 9, piggy-back ports or other ports wherein a needle is inserted for the administration of fluid, medication, solutions and the like.

Although the invention is described as pre-primed IV tubing, the pre-primed tubing could also be in the form of pre-primed piggy-back tubing, which is tubing that is shorter in length than traditional IV tubing.

As described above, the use of the present invention will permit safer delivery of solutions intravenously.

It is to be understood that while a preferred embodiment of the invention is illustrated, it is not to be limited to the specific form or arrangement of parts herein described and shown. It will be apparent to those skilled in the art that various changes may be made without departing from the scope of the invention and the invention is not be considered limited to what is shown and described in the specification and drawings.

Having thus described my invention, I claim:

1. A pre-primed intravenous (IV) tubing comprising:
a predetermined length of IV tubing having a first end and a second end; and
a predetermined solution contained within said IV tubing wherein said predetermined solution is provided in a predetermined volume to expel air bubbles from said IV tubing.

2. The pre-primed intravenous (IV) tubing of claim 1 further comprising:
a cap closing said first end of said IV tubing.

3. The pre-primed intravenous (IV) tubing of claim 2 further comprising:
a cap closing said second end of said IV tubing.

4. The pre-primed intravenous (IV) tubing of claim 1 further comprising:
an IV solution bag containing a predetermined solution; and
said first end of said IV tubing being connected to and in fluid communication with said IV solution bag.

5. The pre-primed intravenous (IV) tubing of claim 4 wherein:
said predetermined solution in said IV solution bag is identical to said predetermined solution in said IV tubing.

6. The pre-primed intravenous (IV) tubing of claim 4 wherein:
said predetermined solution in said IV solution bag is different from said predetermined solution in said IV tubing.

7. The pre-primed intravenous (IV) tubing of claim 4 further comprising:
a cap closing said second end of said IV tubing.

8. The pre-primed intravenous (IV) tubing of claim 5 further comprising:
a cap closing said second end of said IV tubing.

9. The pre-primed intravenous (IV) tubing of claim 6 further comprising:
a cap closing said second end of said IV tubing.

10. The pre-primed intravenous (IV) tubing of claim 1 wherein
said predetermined solution is a universally compatible solution.

11. A pre-primed intravenous (IV) tubing comprising:
a predetermined length of IV tubing having a first end and a second end;
a predetermined solution contained within said IV tubing wherein said predetermined solution is provided in a predetermined volume to expel air bubbles from said IV tubing;
said predetermined solution is a universally compatible solution;
a cap closing said first end of said IV tubing; and
a cap closing said second end of said IV tubing.

12. A method of pre-priming intravenous (IV) tubing comprising a predetermined length of IV tubing having a first end and a second end; and a predetermined solution contained within said IV tubing wherein said predetermined solution is provided in a predetermined volume to expel air bubbles from said IV tubing, said method comprising the steps of:
a. filling said IV tubing with said predetermined solution;
b. expelling air out of said IV tubing; and
c. capping said first end and said second end of said IV tubing for later use.
